(19) United States
(12) Patent Application Publication
(10) Pub. No.: US 2025/0144385 A1
(43) Pub. Date: May 8, 2025

(54) NOVEL AMPOULE FOR DELIVERY OF POLYMERS FOR WOUND TREATMENT

(71) Applicant: Steve Mencanin, Aliso Viejo, CA (US)

(72) Inventor: Steve Mencanin, Aliso Viejo, CA (US)

(21) Appl. No.: 18/935,317

(22) Filed: Nov. 1, 2024

Related U.S. Application Data
(60) Provisional application No. 63/596,197, filed on Nov. 3, 2023.

(51) Int. Cl. A61M 35/00; A61J 1/06; A61J 1/14; A61J 1/20

(52) U.S. Cl. CPC A61M 35/003; A61J 1/067; A61J 1/1412; A61J 1/1443; A61J 1/1468; A61J 1/2024; A61J 1/2093; A61M 2202/0007; A61M 2210/04

(57) ABSTRACT

There is disclosed a wound treatment system having an ampoule with at least two chambers for housing a first and second-containing elastomer component respectively. The ampoule is closed at one end and is sealed at the other end via a tab-seal. Once the tab-seal is removed, a cap is secured to the anterior end of the ampoule via engagement members. The cap is configured with a mixing member found in a channel that runs the length of the cap. As a user applies a lateral force to the ampoule, the first and second-containing elastomer components are mixed by the mixing member to form a skin treatment composition that is applied to the skin of a user for treating a burn, a wound, a scar or a keloid.

### RELATED APPLICATION INFORMATION

This patent claims priority from provisional patent application 63/596,197, filed Nov. 3, 2023, entitled NOVEL AMPOULE FOR DELIVERY OF POLYMERS FOR WOUND TREATMENT, the content of which is included by reference in its entirety.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

### Field

This disclosure relates to chemical delivery and novel methods of treating wounds during times of high stress and where conventional medical treatment is unavailable. More specifically, an ampule loaded with a proprietary polymer which allows mixing of several constituent chemical's when activated is disclosed.

### Description of the Related Art

An ampoule, often spelled “ampul” in some regions, is a small, sealed vial made of glass that contains a sterile solution or medication for injection. Ampoules are used in the healthcare and pharmaceutical industries to store and administer a variety of substances, including vaccines, drugs, and other injectable fluids.

Ampoules are designed to be tamper-proof and airtight, ensuring that the contents remain sterile and uncontaminated until they are ready for use. To access the contents of an ampoule, the neck is usually scored or pre-scored so that it can be easily broken off. Once opened, the contents can be drawn into a syringe for injection or further medical use. Ampoules are often preferred for certain medications and applications because they provide an effective way to maintain the sterility and stability of the contents, and they eliminate the need for preservatives or other additives that may be required in multi-dose vials.

Ampoules made of glass however, limit the type of contents that can be stored. Some chemical make ups require mixing of the chemicals for a final reaction requiring multiple ampoules to store respective chemical mixtures. It further, requires a separate apparatus to combine the chemicals before administering the mixed solution. Administering the final mixture also requires a secure environment, such as a lab or doctor's office, to achieve an accurate mixture. The ability to combine chemicals in an unsecure environment, such as a battlefield or a traumatic situation, with accuracy and efficiency is needed.

### DESCRIPTION OF THE DRAWINGS

FIG. 1 is an upper perspective view of an ampoule.

FIG. 2 is a front view of the ampoule.

FIG. 3 is a left side view of the ampoule.

FIG. 4 is a front cross sectional view of the ampoule.

FIG. 5 is a front perspective view of a cap.

FIG. 6 is a left side view of the cap.

FIG. 7 is an upper perspective view of the cap fastened to the ampoule.

FIG. 8 is a bottom view of the cap.

FIG. 9 is a upper perspective view of a mixing member.

FIG. 10 is a lower perspective cross sectional view of the cap.

FIG. 11 is an upper perspective view of a cap cover.

FIG. 12 is an upper perspective view of a cap cover.

Throughout this description, elements appearing in figures are assigned three-digit reference designators, where the most significant digit is the figure number and the two least significant digits are specific to the element. An element that is not described in conjunction with a figure may be presumed to have the same characteristics and function as a previously-described element having a reference designator with the same least significant digits.

### DETAILED DESCRIPTION

A wound treatment system is herein described. The wound treatment system has an ampoule, a cap and at least two silicone-containing elastomers. The wound treatment system may further have a cap cover.

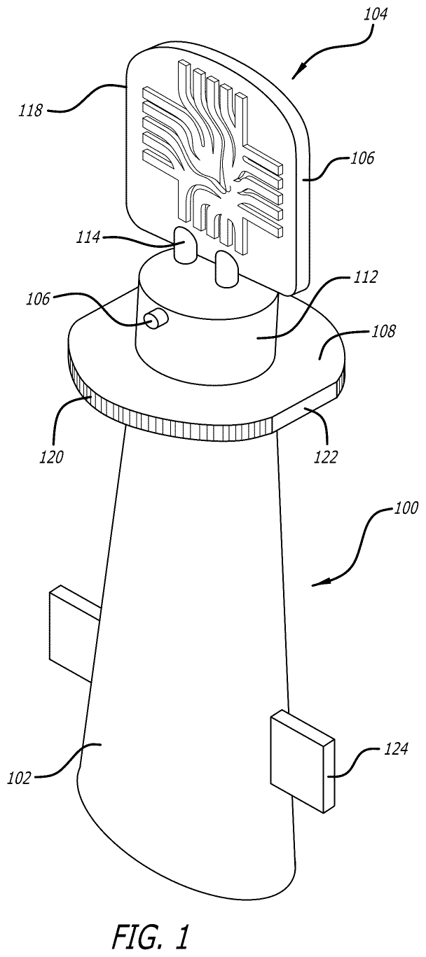

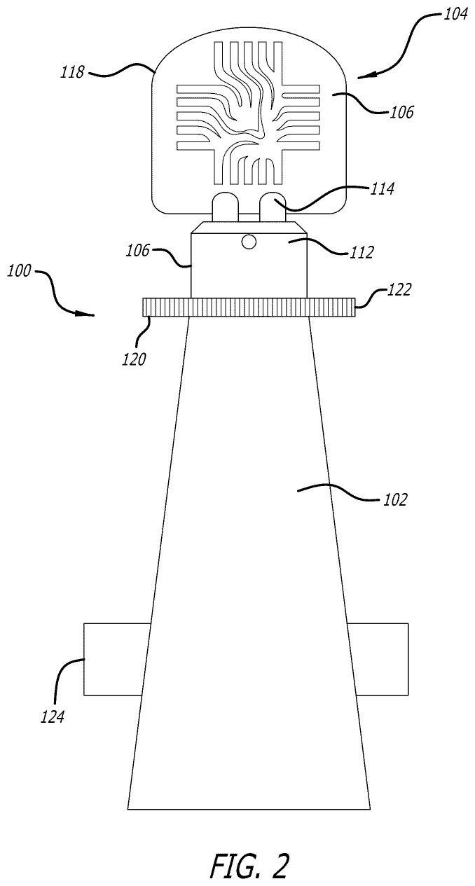

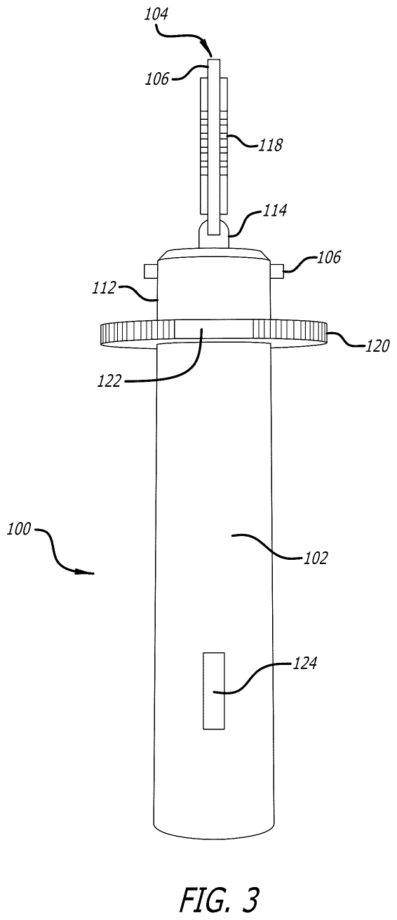

Referring now to FIGS. 1-3, an ampoule 100 is shown. The ampoule 100 is made of a container 102, and a tab-seal 104. The container 102 is formed by a container body 103 and a container top-end 112. The container top-end 112 having at least one engagement member 106 and a collar 108.

The container body 103 has a general tubular shape with a posterior closed end. The container body 103 is not limited to tubular shapes and may have any alternative shape capable of containing contents within. The container body 103 extends upwardly from the closed end and narrows inwardly into the collar 108. The collar 108 expands radially outward from the container body 103 a predetermined distance. From the collar 108 the container top-end 112 extends upwardly a predetermined distance then curves inwardly into a flat surface. The predetermined distances of the collar 108 and the container top-end 113 are dependent on the dimensions as set by the cap (not shown) for which the container top-end 112 is to engage as will be discussed. The volumetric capacity of the container 102 is dependent on the application of the wound treatment contents stored therein. For example, if the wound treatment system is designed to aid with small scrapes or cuts, a smaller container size is needed whereas larger wounds and larger cuts would requires a larger container size to house more of the wound treatment solution.

Removably secured to the container top-end 112 is the tab-seal 104. The tab-seal 104 has at least one crest 114 that is removably secured to the container top-end 112. The crest 114 forms a seal with the container 102 to encapsulate the contents of the container 102 via at least one aperture (not shown) as will be discussed. The crest 114 forms an airtight seal with the container top-end 112 that is broken when the tab-seal 104 is removed exposing the contents of the container 102 to the atmosphere via the aperture (not shown). The crest 114 is configured such that sufficient radial/lateral exertion (twisting force) upon the tab-seal 104 will break the seal formed by the crest 114 and the container top-end 112. From the crest 114, a tab 116 extends laterally outward and then extends upwardly some distance before curving inwardly toward the distal end of the tab 116. The tab 116 serves as a pull tab for users to grip when removing the tab-seal 104 from the container top-end 112. For example, when a user is attempting to remove the tab-seal 104 the user will grip the tab 116 on either side and exert a twisting force to break the seal between the crest 114 and the container top-end 112.

The tab-seal 104 may be configured with plugs (not shown) in lieu of the crests 114. In such a manner, the tab-seal 104 may be used to reseal the container 102. The plugs would be removably secured to the container top-end 112 such that an airtight seal is formed when the plugs are engaged with the apertures. The user may then proceed with the application of the wound treatment system as described in this disclosure, and then may put back the tab-seal 104 to store unused contents of the container 102. A benefit of utilizing plugs for the tab-seal is the reusability of the container 102. For example, the container 102 may have a larger volumetric capacity for multiple uses, wherein the plugs prevent the contents stored in the container from mixing/leaking between uses. In this way, the container 102 may be more cost effective because less ampoule units would be wasted between treatments without losing sterility. The tab-seal 104 may also be integrally formed with the container 102 via a hinge, such that the tab-seal 104 may freely dangle from the container when applying the contents of the container 102 during use.

The tab 116 may take the form of various shapes such as but not limited to: a sphere, a rectangle, or any desirable shape such that a user may take hold of the tab-seal 104 and exert a twisting force to break the seal formed by the crest 114 and the container top-end 112. The tab 116 may also have protrusions 118 that extend outwardly from the exterior of the tab 116 to aid with tactile grip. For example, if a user in a combat setting may have blood on their hands when aiding a wounded person and can use the protrusions 118 to aid with traction to remove the tab-seal 104 from the container 110.

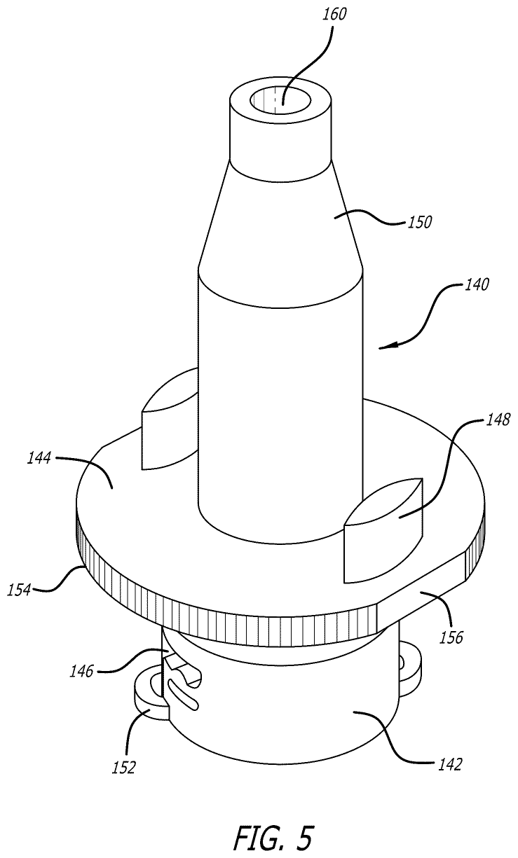

Located on the peripheral edge of the container top-end 112 is the engagement member 106. In the present disclosure, there are two engagement members 106 located on the front and rear of the container top-end 112. The engagement members 106 protrude outwardly from the container top-end 112 a predetermined distance and is located a predetermined distance from the collar 108. The predetermined distances of the engagement members are determinative of the cap's (not shown) dimensions for which the engagement member 106 is to engage as will be discussed. The engagement member 106 is formed to securely engage with a cap as discussed later in this disclosure (see FIG. 5). The predetermined distance is determinative of the cap (not shown) dimensions for which is to engage with the engagement members 106 as will be discussed. As such, the location and number of the engagement members 106 is respective to the cap. For example, if the cap requires one or more engagement members 106 to securely fasten the ampoule 100, the engagement members 106 will be dispersed about the container top-end 112 respectively. The engagement member 106 has a general cylindrical shape but may have any shape capable of securing/engaging with the cap.

The collar 108 supports the cap (not shown) when it is securely fastened to the container top-end 112 via the engagement member 106. The collar 108 is integrally formed with the container top-end 112 extending radially outward from the container body 103. The collar 108 has a plurality of ridges 120 along it's peripheral edge. The benefit of the plurality of ridges 120 is to provide additional grip for a user when securing the cap to the ampoule 100. Further, the collar 108 may have a circular segment 122 on the lateral sides of the collar 108. The benefit of the circular segment 122 is to aid with gripping the ampoule 100 and may aid with the manufacturing of the ampoule 100. For example, the circular segment 122 may serve as a connection point for manufacturing molded ampoules 100.

Extending from the lateral side of the container body 103 are a set of tabs 124. The set of tabs 124 are integrally formed with the container body 103. The set of tabs 124 extend outwardly from the container body 103 and have a general rectangular shape of a predetermined thickness. The set of tabs 124 may assist the user with gripping the ampoule 100 when fastening the cap or serve as structural support during the manufacturing process of the ampoule. The set of tabs 124 may have any alternative shape and of varying thickness capable of providing sufficient structural support and/or provide a user the ability to adequately grip the set of tabs 124.

The ampoule 100 is made of a pliable material such as plastic. The ampoule may be made from such plastics such as but not limited to: polypropylene, polyethylene, polycarbonate or any other type of plastic capable of mixing with the at least two different chemicals as disclosed. The benefits of utilizing a plastic material for the ampoule 100 is increased durability of the ampoule 100 during transportation. For example, common forms of ampoules are formed of glass which limits the applicable settings for use. In using a plastic material, the ampoule 100 is less likely to break, and thus preserves the contents of the ampoule 100 for its intended use. Furthermore, the increased durability in using plastic permits the ampoule 100 to be squeezed without permanent deformation when a user is applying a wound treatment.

The ampoule 100 may be manufactured using one of numerous techniques such as but not limited to: injection molding, compression molding, blow molding, thermoforming, transfer molding, reaction injection molding or any methods of forming plastic into the ampoule 100 as described in the present disclosure.

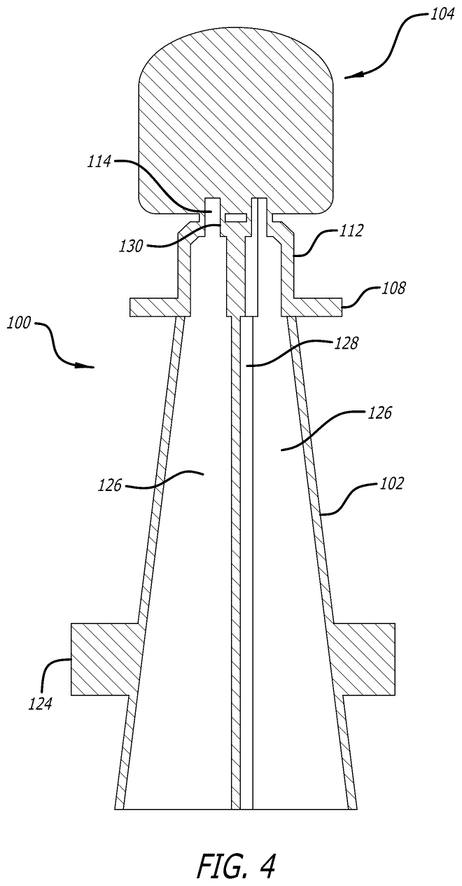

As shown in FIG. 4, the container top-end 112 and the collar 108 may be configured as one piece such that the collar 108 extends into the container top-end 112. The container top-end 112 and the collar 108 are formed of the same thickness that is greater than the thickness of the container body 103. The benefits of having a greater thickness for the container top-end 112 and the collar 108 is increased durability for when engaging with the cap (not shown). For example, when a user engages the ampoule 100, the container top-end 112 is to withstand the compressive force from a user squeezing the container body 103 to remove the contents of the container 102. Further, the cap is to engage with the container top-end 112 and the collar 108, requiring that they withstand the engagement with the cap. For example, as will be discussed, the cap is removably engaged with the container top-end 112 via engagement members, in this way the thicker material of the container top-end 112 and the collar 108 prevent deformation of the same when the user fastens the cap. The container top-end 112 is formed in the shape of a cylinder having a hollow interior that extends from the thinner walls of the container body 103. The container body 103 extends from the bottom of the container 102 up to the base of the collar 108. The container top-end 112 has a smaller diameter than the container body 103 such that the base of the collar 108 leaves a beveled edge where the container body 103 meets the collar 108. The container top-end 112 then extends cylindrically upward into the substantially flat end where at least two apertures 130 is bored through. A benefit of the container top-end 112 having a smaller diameter than the container body 103 is the increase flow rate of the contents stored in the container 102 as they exit the container 102. In this way, the contents being squeezed out of the container 102 experience a difference in pressure as the volumetric capacity of the container 102 decreases in the direction in which the contents exit. The lateral force applied to the container body 103 force the contents stored in the container 102 to be pushed upward through the apertures 130. The smaller diameter in the container top-end 112 from the container body 103 further increases pressure and subsequently the flow rate. An increase in flow rate helps in mixing the contents stored in the container 102 as they exit the container 102 as will be discussed.

The container 102 is configured to have at least two chambers 126. The chambers 126 are formed by a partitioning wall 128 that extends from the container top-end 112 downward toward the closed posterior end of the container 102. The partitioning wall 128 extends laterally to the side walls of the container 102, thus forming at least two chambers 126. It is to be understood that there may be a plurality of partitioning walls 128 such that a plurality of chambers 126 may be formed within the container 102. For example, there may be two partitioning walls 128 that extend laterally to the sides of the container 102 forming a cross shape, thus forming four separate chambers 126. The partitioning wall 128 further differs in thickness, respective to where the partitioning wall 128 is positioned within the container 102. For example, the partitioning wall 128 is thicker when extending through the container top-end 112 and tapers into a thinner portion when extending through the container body 103. The aperture 130 is located on the flat surface of the container top-end 112. The number of apertures 130 is respective to the number of chambers 126 in the container 102. For example, if the container 102 has two chambers 126 then there are two apertures 130, or if there are three chambers 126 then there are three apertures 130. The apertures 130 are centrally located to the respective chambers 126. The aperture 130 are formed after the removal of the tab-seal 104. The crest 114 of the tab-seal 104 is formed with the container top-end 112 such that the lateral walls of the crest 114 circumscribe the diameter of the apertures 130. In this way, when the tab-seal 104 is removed from the container 102, the crest 114 breaks away from the container top-end 112 along the peripheral edge forming the apertures 130. The crest 114 are generally hollow to reduce the amount of material needed to seal the apertures 130. A benefit of forming the seal this way is to provide ease of removing the tab-seal 104, such that the contents of the ampoule 100 may freely exit the chambers 126 without obstruction. For example, traditional ampoules formed of glass are broken along a perforated edge wherein glass particles may mix with the contents. The reduced material in the crest 114 allows for a user to shear the tab-seal 104 from the container top-end 112 such that no material mixes with the contents stored within the chambers 130. Further, the container top-end 112 having a thickness greater than the crest 114 permits container top-end 112 to withstand the shear force applied when removing the tab-seal 104.

Further, the crest 114 forms an air-tight seal with the aperture 130, such that the contents of the chambers 126 cannot escape. It is to be understood that the number of crests 114 is respective to the number of apertures 130 of the ampoule 100. For example, if the ampoule 100 has two chambers 126 then there are two apertures 130, and thus two crests 114 or if there are three chambers 126 then there are three apertures 130 and thus three crests 114.

The benefits of having numerous chambers 126 is the ability for the ampoule 100 to store various chemicals within each respective chamber 126. In the present embodiment, by including at least two chambers 126, two different chemicals can be stored in each of the respective chambers 126. The benefits of storing and separating chemicals in the chambers 126 is that it reserves a polymerization reaction to take place in real time. For example, the stored chemicals described herein may be used to treat a wound, and if the chemicals were to polymerize before applying it to a user's wound, there would be a loss in effectiveness. Instead, the ampoule 100 permits a user to cause the polymerization on location upon an would be applicant's skin, such as on the battlefield.

Polymerization reactions are chemical processes in which small molecules, called monomers, join together to form larger, more complex molecules known as polymers. Polymers are composed of repeating structural units, which are essentially the monomers linked together in a chain or network. Polymerization reactions can be broadly categorized into two main types: addition (chain-growth) polymerization and condensation (step-growth) polymerization.

Several appropriate polymerization reactions are discussed below. Addition (Chain-Growth) Polymerization. Monomers: In this type of polymerization, monomers with unsaturated bonds (typically double bonds) are used. Common examples include ethylene, propylene, and styrene. Initiation: The polymerization reaction begins with the initiation step, where a chemical initiator, such as a free radical, is introduced. Initiators are compounds that can break the double bond in the monomers, creating highly reactive species called free radicals. Propagation: During the propagation step, the free radicals generated in the initiation step attack the double bond of other monomers, causing them to join the growing polymer chain. This process repeats as more and more monomers add to the chain, making it longer. Termination: Termination can occur when two free radicals combine or when a free radical reacts with an inactive species, effectively ending the growth of the polymer chain.

Another appropriate polymerization reaction includes Condensation (Step-Growth) Polymerization. Monomers: In condensation polymerization, monomers contain functional groups that can react with each other by forming covalent bonds. Common examples include polyesters and polyamides. Reaction: In this process, two different monomers or a bifunctional monomer react with each other. When the monomers combine, a small molecule, typically water, is eliminated as a byproduct. Repeating units: The reaction continues until all the functional groups on the monomers have reacted, forming a long polymer chain with repeating units. End groups: The resulting polymer may have end groups left from the original monomers or other byproducts, depending on the specific reaction conditions.

A multitude of factors and chemicals were considered when determining S-polymer was the appropriate compound to include within the S-ampoule. Please note S-ampoule along with present disclosure are used to refer to the ampoule and method of producing ampoule discussed through this disclosure.

Monomers Selection: The first step is to select two different monomers, each containing reactive functional groups. These functional groups can react with each other to form covalent bonds. The choice of monomers determines the type of polymer that will be formed and its properties. S-polymer and the monomers that are used to create it was selected because S-polymer can harden in an environment where blood and human tissue is present. More importantly the monomers making up S-polymer do not react with other chemicals within the blood nor do they affect the buffer system or pH of blood. This is crucial to the present disclosure because other monomers may form polymers with chemicals within the blood resulting in blood clots and death of the user.

Combining Monomers: The selected monomers are mixed together. In the reaction, the functional groups on one monomer interact with the functional groups on the other monomer, leading to the formation of covalent bonds between the monomers. Typically, one monomer contains two reactive groups (bifunctional monomer), and the other monomer also contains two reactive groups. Polydimethylsiloxane is an appropriate polymer for the presently disclosed Ampoule The following monomers derived from the below chemical are also appropriate:

[—Si(CH3)2—O—]n

In this representation, n represents the number of repeating units, and each unit consists of a silicon atom (Si) bonded to two methyl groups (CH3) and two oxygen atoms (O). These units are linked together through silicon-oxygen-silicon (Si—O—Si) bonds.

S-Polymer is an ideal choice for several reasons. Biocompatibility: PDMS is known for its biocompatibility. It has been extensively used in medical and biological applications for many years, including in devices like catheters, artificial organs, and medical implants. It is generally well-tolerated by the human body and doesn't trigger significant immune responses or adverse reactions. Inertness: The Si—O—Si bonds in PDMS create a highly inert material. Inert materials do not readily interact with other substances, including blood. This inertness helps prevent the material from undergoing chemical reactions or degradation when exposed to biological fluids.

In other embodiments S-Polymer may take the form of Diphenylmethane diisocyanate. Some sources including the SDS for Cyanoacrylate indicate that Cyanoacrylate should not be used on the skin. However, in the experience of the inventors Cyanoacrylate only caused eye and respiratory harm, but does not harm the epidermis.

Stability: Silicones, in general, are known for their stability over a wide range of environmental conditions, including exposure to bodily fluids. The Si—O—Si bonds are robust and do not break down easily in the presence of blood or other biological fluids, ensuring the integrity of the material. Low Surface Energy: PDMS has a low surface energy, which means it is not very “sticky” and does not tend to interact strongly with molecules in the blood. This characteristic helps prevent protein adsorption and cell adhesion, which can be beneficial in certain medical applications. Hydrophobic Nature: PDMS is hydrophobic, meaning it repels water. While blood contains water, the hydrophobic nature of PDMS helps keep it separate from the material, reducing the risk of undesirable interactions.

Elimination of Small Molecules: As the monomers react and form covalent bonds, a small molecule is often eliminated as a byproduct. For example, in the formation of a polyester, water (H2O) is typically produced as a byproduct. This byproduct is often a molecule with one atom from each of the reacting monomers.

Repeating Units: The reaction continues until all of the functional groups on the monomers have reacted. The result is a polymer with a chain of repeating structural units, which are covalently bonded to each other. These repeating units form the backbone of the polymer chain. End Groups: The resulting polymer may still have end groups left from the original monomers, which are typically unreacted functional groups. The presence of these end groups may affect the properties of the polymer, and sometimes they are modified or removed in post-polymerization steps. Polymerization Conditions: The specific conditions under which the reaction is carried out, such as temperature, pressure, and the presence of catalysts or initiators, can influence the rate of polymerization and the properties of the resulting polymer. Chain length and structure: The length and structure of the polymer chain depend on factors like the monomer's reactivity, the type of initiator used, and reaction conditions.

In the present embodiment, each chamber 126 of the ampoule 100 is intended to house a silicone containing elastomer, that when combined allow for polymerization to form a skin treatment composition. The skin treatment composition, comprises: a solution for forming a first layer over damaged skin, the solution comprising: isopropyl alcohol, colloidal silver, chamomile extract, and gotu kola extract; a silicone mixture for forming a second layer over the first layer, the silicone mixture comprising: polydimethylsiloxane, noncrystalline silicone dioxide, a methylhydride crosslinker, a platinum catalyst, and hydroxyl terminated polymethylphenylsiloxane; and a mineral powder for forming a third layer over the second layer, the mineral powder comprising: mica and a natural mineral kaolinite. The skin treatment composition may be applied to a user's burn, wound, scar and/or a keloid on the user's skin.

The skin treatment composition may have an alternative composition wherein one chamber 126 has a first silicone elastomer including: 50-80% by wt. of a polydimethylsiloxane, 10-25% by wt. of a noncrystalline silicone dioxide, 0.03-0.09% by wt. of a platinum catalyst, and 0.18-0.49% by wt. of a hydroxyl terminated polymethylphenylsiloxane; and another chamber 126 has a second silicone elastomer including: 50-80% by wt. of a polydimethylsiloxane, 10-25% by wt. of a noncrystalline silicone dioxide, no more than 10% by wt. of a methylhydride crosslinker, and 0.18-0.49% by wt. of a hydroxyl terminated polymethylphenylsiloxane, wherein the first and second silicone elastomers are configured to react when mixed in order to form a film layer for treating a skin area.

In alternate embodiments of the ampoule 100 having numerous chambers 126, the ampoule 100 may store a solution in a first chamber, the solution including: 0.5-3% by wt. of an isopropyl alcohol, 73-90% by wt. of a colloidal silver, 1-5% by wt. of a chamomile extract, and 1-5% by wt. of a gotu kola extract; a second chamber 126 holding a first silicone elastomer, the first silicone elastomer including: 50-80% by wt. of a polydimethylsiloxane, 10-25% by wt. of a noncrystalline silicone dioxide, 0.03-0.09% by wt. of a platinum catalyst, and 0.18-0.49% by wt. of a hydroxyl terminated polymethylphenylsiloxane; and a third chamber 126 holding a second silicone elastomer, the second silicone elastomer comprising: 50-80% by wt. of a polydimethylsiloxane, 10-25% by wt. of a noncrystalline silicone dioxide, no more than 10% by wt. of a methylhydride crosslinker, and 0.18-0.49% by wt. of a hydroxyl terminated polymethylphenylsiloxane; and a fourth chamber 126 having a mineral powder, the mineral powder including 3-5% by wt. of a natural mineral kaolinite.

Each of the described embodiments that house various contents in respective chambers 126 of the ampoule 100 involve the process of removing the tab-seal 104 from the container 102, depressing the container 102 such that each of the respective contents are free to exit the container 102 to combine through polymerization. The process includes adequate mixing of the components through the cap as will now be described.

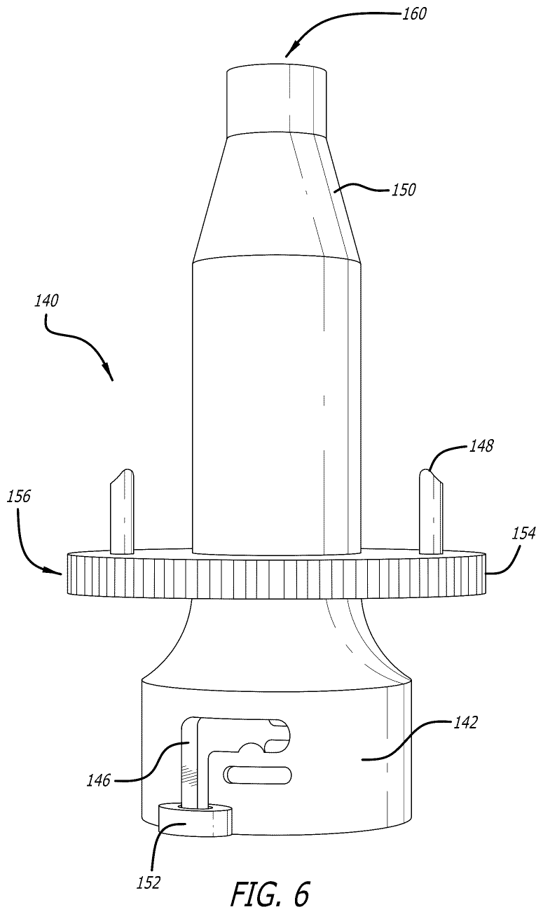

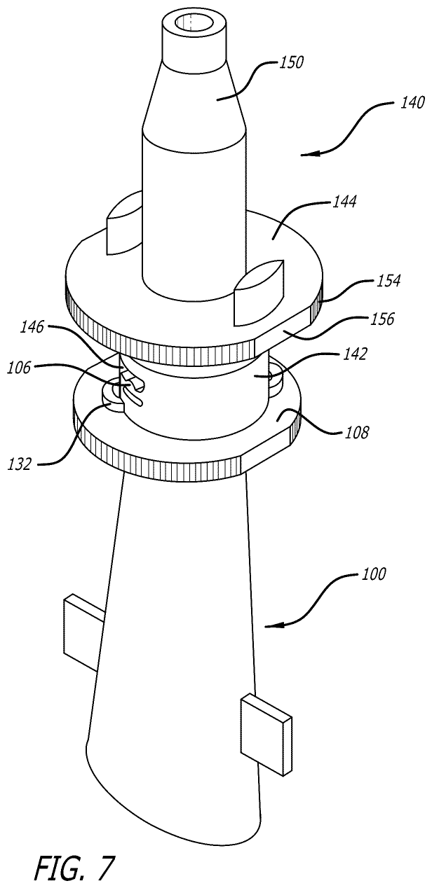

Referring now to FIGS. 5-6, a cap 140 is shown. The cap 140 has a cap base 142, a cap collar 144, a locking groove 146, a cap engagement member 148, a channel 160 and a cap tip 150. The cap base 142 is configured to engage with the container top-end such that the container top-end (see FIG. 7) sits entirely within the cap base 142. In this way, the cap base 142 extends radially upward a predetermined length to the top side of the container base 142 having a hollow interior. The cap base 142 having an inner diameter respective to the container top-end. The cap base 142 then extends into the cap tip 150. The cap tip 150 is of a smaller diameter than the cap base 142 and extends radially upward a predetermined length before contracting inwardly at a final diameter at the distal end of the cap tip 150. The cap tip 150 has a hollow interior such that the channel 160 is formed by the hollow interior of the cap base 142 and the cap tip 150. The predetermined length of the cap tip 150 is of sufficient length to house a mixing member (not shown) in the channel 160 as will be discussed, and to permit adequate mixture of the stored contents in the ampoule.

As shown in FIG. 7, the locking groove 146 is formed by a bored path in the cap base 142 that is configured to engage with the engagement member 106 of the ampoule. The locking groove 146 has at least one engagement tab 152 that protrudes outwardly from the cap base 142 to guide the engagement member 106 into the locking groove 146. The engagement tab 152 is configured to engage with the engagement member 106 such that it may be in the shape of a circle, rectangle, or any other shape to configure with the engagement tab 152.

Referring back to FIG. 6, the locking groove 146 is bored through the cap base 142 extending upwardly a predetermined distance such that the container top-end 112 is secured within the interior of the cap base 142, and the bottom of the cap base 142 is engaged with the top surface of the collar of the ampoule. The locking groove 146 then turns at a right angle in order to securely fasten the engagement member 106 into the locking groove 146. For example, after a user has removed the tab-seal from the ampoule, the user will then align the engagement tabs 152 with the engagement member and apply a downward force, then a rotational force, to securely fasten the cap 140 to the ampoule. The benefits of having a locking groove 146 configured in the cap 140 is to ensure the cap 140 does not disengage from the ampoule before the contents of the ampoule have adequately mixed.

The cap 140 may be made of plastic or metal or other material that can be sterilized, such that the cap 140 can be re-used for other applications. For example, it may be cost effective for the ampoule to be made from plastic, and the cap 140 made from a re-usable material, such as metal, such that after use, the ampoule is disposed of but the cap 140 is sterilized for the next use. Furthermore, the cap 140 may also be configured to use a cap cover, as will be discussed, such that the cap cover and ampoule may be disposed of after use but a re-usable cap 140 can be interchanged between uses. For example, a user may apply the contents of a first ampoule using a cap 140 with a first cover to one user and then interchange the first cap cover and ampoule for a second unused cap cover and ampoule using the same cap 140, then apply the treatment to a second application while maintaining the same cap 140 with safety and sterility.

In other embodiments, the cap 140 may be integrally formed with the ampoule 100 such that the cap 140 is securely fastened to the ampoule 100 via a hinge. In this way, the cap 140 and the ampoule are an all-in-one system. For example, once the tab-seal is removed from the ampoule, the user can immediately secure the cap 140 to the ampoule. The benefits of an all-in-one system is ease of use and increased efficiency during deployment of the wound treatment. In alternative embodiments, the cap 140 may be press fit atop the ampoule 100 such that the engagement member and locking groove 146 may be omitted. In this embodiment, the container may also have a locking tab to engage with an indentation in the cap 140 such that the cap 140 locks into place when the locking tab and indentation marry. The cap 140 may also be configured with the tab-seal such that when the user twists off the cap, the tab-seal disengages with the container. The tab-seal may then be removed from the cap 140, and the cap 140 can then be placed atop the ampoule as presently described.

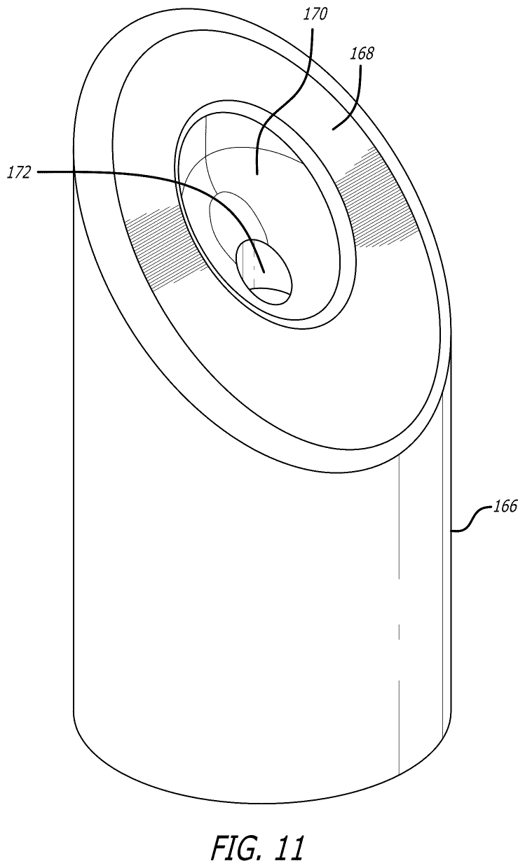

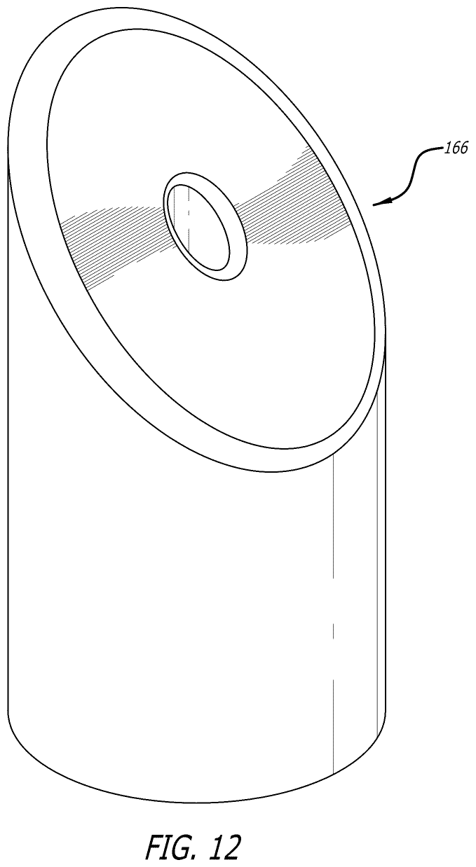

Referring to FIGS. 5-7, the cap collar 144 has a plurality of ridges 154 along it's peripheral edge. The benefits of the plurality of ridges 154 is to provide additional grip for a user when securing the cap 140 to the ampoule 100 or securing a cap cover to the cap 140 as will be discussed. Further, the cap collar 144 may have a circular segment 156 on the lateral sides of the cap collar 144. The benefits of the circular segment 156 is to add additional grip and aid with the manufacturing process of the cap 140. For example, the circular segment 156 may serve as a connection point for molded caps 140. The cap engagement member 148 is formed with the cap collar 144 as a protrusion extending upwardly from the cap collar 144. The cap engagement member 148 is configured to fasten the cap cover (see FIG. 11-12) over the cap tip 150. The cap engagement member 148 may press fit the cap cover to the cap 140 or may be configured to fasten the cap cover through sufficient means such as threads, or lock and groove.

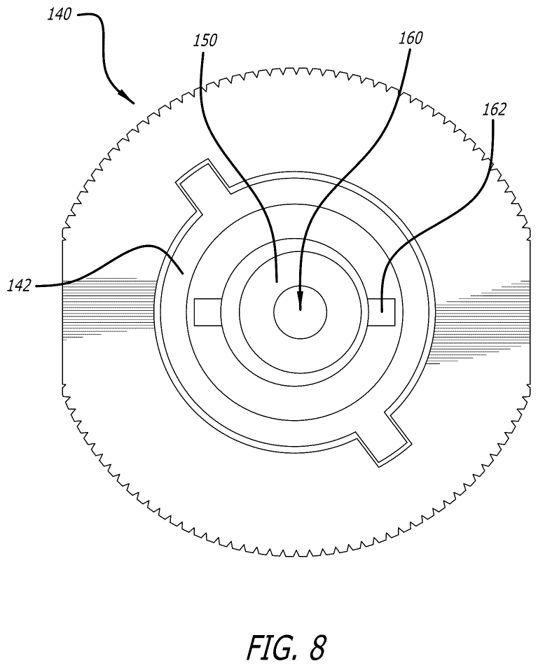

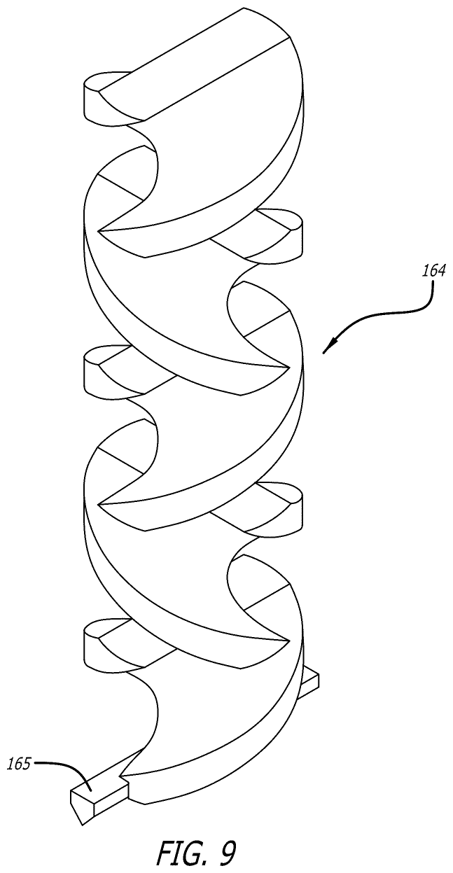

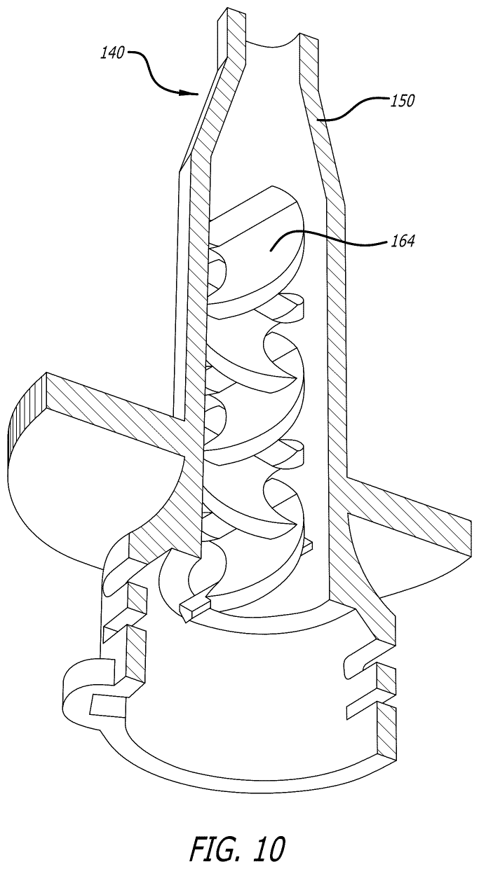

Referring now to FIG. 8, at least one mixing groove 162 is located on the topside of the cap base 142 interior. The purpose of the mixing groove 162 is to removably secure a mixing member 164, as shown in FIG. 9, to the channel 160 of the cap 140. The mixing member 164 has engagement members 165 that mate with the by the mixing grooves 162. As shown in FIG. 10, the mixing member 164 sits within the channel 160 of the cap 140. It is to be understood that the mixing member 164 may also be permanently secured to the cap 140 such as with glue, or molded together or other sufficient methods to permanently secure the mixing member 164 to the cap 140. The mixing member 164 has a length respective to the cap tip 150 such that adequate mixing of the contents of the ampoule may occur before exiting the cap 140 into the atmosphere. The mixing member 160 is formed by a helical spiral. The benefit of using a helical spiral is to ensure adequate mixture of the contents within the ampoule. For example, the ampoule may have two silicone elastomer's stored in respective chambers, such that when the tab-seal of the ampoule is removed, the cap 140 with the mixing member 160 fastened therewithin is secured to the ampoule, a user will then exert a lateral force on the ampoule causing the respective silicone elastomer's to be forced upwardly from their chambers and into the cap, the respective silicone elastomers will then pass over the helical grooves of the mixing member 160 for adequate polymerization to form the skin treatment composition before exiting the cap 140 and onto the skin of a would be applicant. The mixing member 160 may have various shapes that provide for adequate mixing of the contents stored within the ampoule.

The cap 140 and the mixing member 164 may be formed of the same or different material including but not limited to: polypropylene, polyethylene, polycarbonate, aluminum, steel, glass or any other type of material capable of performing the functions of the cap 140 and the mixing member 160 as described herein. Further, the mixing member 160 may also be made from a reusable material that can be sterilized for multiples uses.

Referring now to FIG. 11, a cap cover 166 is shown. The cap cover 166 is configured to be removably secured to the cap via the cap engagement members. The cap cover 166 is hollow and has an angled flat face 168 and further has a recess 170 central to the angled flat face 168. The cap cover 166 has an aperture 172 centrally located to the angled flat face 168 that permits the skin treatment composition to exit from the cap tip. The cap cover 166 alters the way the skin treatment composition applies to a user's skin. For example, when the cap cover makes contact with the skin of a would be applicant, the skin treatment composition exit the cap cover 166 and pools within the recess 168, the user can then moves over the skin of the user wherein the skin treatment composition spread about the angled flat face 168 and is spread evenly onto the applicant's skin. FIG. 12 shows an alternative embodiment of the cap cover 166 having only an angled flat face and an aperture as described in FIG. 11.

### Closing Comments

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A wound treatment system comprising:
an ampoule having a first chamber and a second chamber formed by an internal partition that extends from the posterior end of the ampoule to the anterior end of the ampoule, the posterior end of the ampoule is closed, the anterior end of the ampoule is formed by a top-end having a collar that extends radially outward from the ampoule, located atop the top-end is a first and second aperture respective to the first and second chambers, the first and second apertures are sealed using a removable tab-seal configured to enclose the first and second apertures of the first and second chambers, extending outwardly from the top-end is least one engagement member, and extending outwardly from the body of the container is a set of tabs;
a first silicone-containing elastomer component is housed in the first chamber;
a second silicone-containing elastomer component is housed in the second chamber;
a cap a cap having at least one cap engagement member and a mixing member, the cap configured to engage with the at least one engagement member via a locking groove formed in the base of the cap, wherein the cap is formed by a channel that extends the length of the cap, the cap having a base configured to be seated atop the top-end of the ampoule, the mixing member is removably secured within the channel via at least one mixing groove, a cap collar that extends radially outward from the cap, and at least one cap engagement member located about the cap collar;
wherein, upon securing the cap to the ampoule and applying a lateral force to the exterior of the ampoule, the first and second silicone-containing elastomer components are pushed outwardly from the first and second chambers through the cap, the first and second silicone-containing elastomer components are mixed to form a skin treatment composition before exiting the open end of the cap.

2. The wound treatment system of claim 1, wherein the skin treatment composition are applied to a user's skin for treating at least one of a burn, a wound, a scar or a keloid.

3. The wound treatment system of claim 1, the first silicone-containing elastomer component comprises between 0.03-0.09 wt % of a silicone platinum catalyst, between 50-80 wt % of a polydimethylsiloxane, between 10-25 wt % of a noncrystalline silicone dioxide, and between 0.18-0.49 wt % of a hydroxyl terminated polymethylphenylsiloxane.

4. The wound treatment system of claim 1, wherein the second silicone-containing elastomer component comprises no more than 10 wt % of a crosslinker, between 50-80 wt % of a polydimethylsiloxane, between 10-25 wt % of a noncrystalline silicone dioxide, and between 0.18-0.49 wt % of a hydroxyl terminated polymethylphenylsiloxane.

5. The wound treatment system of claim 1, wherein the collar has a plurality of ridges aligning the peripheral edge of the collar.

6. The wound treatment system of claim 1, wherein the collar has a circular segment bisecting the lateral sides of the collar.

7. The wound treatment system of claim 1, wherein the cap collar has a plurality of ridges aligning the peripheral edge of the collar.

8. The wound treatment system of claim 1, wherein the cap collar has a circular segment bisecting the lateral sides of the collar.

9. The wound treatment system of claim 1, wherein the mixing member is formed by a helical spiral.

10. The wound treatment system of claim 1, wherein the tab-seal has protrusions extending from the exterior body of the tab-seal.

11. The wound treatment system of claim 1, wherein the ampoule formed of a pliable material.

12. The wound treatment system of claim 1, wherein a cap cover is removably secured to the cap via the at least one cap engagement member.

13. A method for treating at least one of a burn, a wound, a scar and a keloid on a user's skin, comprising:
removing a tab-seal from an ampoule, the tab-seal sealing a first and second chamber of the ampoule, exposing a respective aperture of the first and second chambers;
fastening a cap atop the ampoule and the respective apertures of the first and second chambers via at least one engagement members of the ampoule;
applying a lateral force to each of the first and second chambers to secrete a first silicone-containing elastomer component from the first chamber and a second silicone-containing elastomer components from the second chamber;
the first silicone-containing elastomer component comprising:
between 0.03-0.09 wt % of a silicone platinum catalyst;
between 50-80 wt % of a polydimethylsiloxane;
between 10-25 wt % of a noncrystalline silicone dioxide; and
between 0.18-0.49 wt % of a hydroxyl terminated polymethylphenylsiloxane;
the second silicone-containing elastomer component comprising:
no more than 10 wt % of a crosslinker;
between 50-80 wt % of a polydimethylsiloxane;
between 10-25 wt % of a noncrystalline silicone dioxide; and
between 0.18-0.49 wt % of a hydroxyl terminated polymethylphenylsiloxane;
mixing the first and second silicone-containing elastomer components through a mixing member integrated in the cap;
applying the mixed first and second silicone-containing elastomer components to the at least one of the burn, the wound, the scar and the keloid on the user's skin to create an elastomer gel that forms, within ten minutes, a film having a thickness and tensile strength effective to compress the user's skin and decrease formation of fibroblasts;
wherein at least one of the first and second components comprises between 0.49-0.98 wt % of amorphous fumed silica; and
wherein the film is water resistant, provides a protective layer to the user's skin, and can be peeled off as a single piece.

14. A wound treatment system comprising:
an ampoule having a plurality of chambers,
the plurality of chambers are formed by a partition respective to the number of the plurality of chambers that extend from the posterior end of the ampoule to the anterior end of the ampoule and extend the length of the lateral walls of the ampoule, the plurality of chambers are sealed at one end, and have a plurality of apertures respective to the number of the plurality of chambers on the opposite end, the anterior end of the ampoule further having at least one engagement members and a collar;
the plurality of chambers are sealed by a removable tab-seal;
a silicone-containing elastomer component is housed in each of the plurality of chambers;
a cap configured to engage with the engagement members after removing the tab-seal, the cap having a mixing member configured within a channel of the cap,
wherein, upon applying a lateral force to the exterior of the ampoule, the silicone-containing elastomer components are pushed outwardly from the plurality of chambers through the cap and the silicone-containing elastomer components are mixed together to form a skin treatment composition before exiting the cap.

15. The wound treatment system of claim 14, wherein the skin treatment composition are applied to a user's skin for treating at least one of a burn, a wound, a scar or a keloid.

16. The wound treatment system of claim 14, the first silicone-containing elastomer component comprises between 0.03-0.09 wt % of a silicone platinum catalyst, between 50-80 wt % of a polydimethylsiloxane, between 10-25 wt % of a noncrystalline silicone dioxide, and between 0.18-0.49 wt % of a hydroxyl terminated polymethylphenylsiloxane.

17. The wound treatment system of claim 14, wherein the second silicone-containing elastomer component comprises no more than 10 wt % of a crosslinker, between 50-80 wt % of a polydimethylsiloxane, between 10-25 wt % of a noncrystalline silicone dioxide, and between 0.18-0.49 wt % of a hydroxyl terminated polymethylphenylsiloxane.

18. The wound treatment system of claim 14, wherein the ampoule formed of a pliable material.

19. The wound treatment system of claim 14, wherein the ampoule further comprises a set of tabs extending outwardly from the lateral sides of the ampoule.

20. The wound treatment system of claim 14, wherein the mixing member is formed by a helical spiral.

21. A wound treatment system comprising:
an ampoule having a first chamber and a second chamber formed by an internal partition that extends the length of the ampoule, the posterior end of the ampoule is closed, the anterior end of the ampoule is formed by a top-end having a first and second aperture configured to engage with the first and second chambers, extending outwardly from the peripheral edge of the top-end is least one engagement member, the first and second apertures are sealed using a tab-seal configured to enclose the first and second apertures, the tab-seal formed by two plugs and a tab, he two plugs are configured to engage with the first and second apertures to create a seal;
a first silicone-containing elastomer component is housed in the first chamber;
a second silicone-containing elastomer component is housed in the second chamber;
a cap having at least one cap engagement member and a mixing member, the cap is configured to engage with the at least one engagement member via a locking groove formed in the base of the cap, wherein the cap is formed by a channel that extends the length of the cap, the mixing member is formed by a helical spiral and is removably secured within the channel via at least one mixing groove, the cap configured to be seated atop the top-end of the ampoule;
a cap cover configured to engage the cap engagement member;
wherein, upon securing the cap to the ampoule and applying a lateral force to the exterior of the ampoule, the first and second silicone-containing elastomer components are pushed outwardly from the first and second chambers through the cap, the first and second silicone-containing elastomer components are mixed to form a skin treatment composition before exiting the open end of the cap.

22. The wound treatment system of claim 1, wherein the skin treatment composition are applied to a user's skin for treating at least one of a burn, a wound, a scar or a keloid.

23. The wound treatment system of claim 1, the first silicone-containing elastomer component comprises between 0.03-0.09 wt % of a silicone platinum catalyst, between 50-80 wt % of a polydimethylsiloxane, between 10-25 wt % of a noncrystalline silicone dioxide, and between 0.18-0.49 wt % of a hydroxyl terminated polymethylphenylsiloxane.

24. The wound treatment system of claim 1, wherein the second silicone-containing elastomer component comprises no more than 10 wt % of a crosslinker, between 50-80 wt % of a polydimethylsiloxane, between 10-25 wt % of a noncrystalline silicone dioxide, and between 0.18-0.49 wt % of a hydroxyl terminated polymethylphenylsiloxane.

25. The wound treatment system of claim 21, wherein the top end has a collar extending radially outward therein.

26. The wound treatment system of claim 25, wherein the cap collar has a plurality of ridges aligning the peripheral edge of the collar.

27. The wound treatment system of claim 21, wherein the ampoule formed of a pliable material.

28. The wound treatment system of claim 21, wherein the cap formed of a sterile material.

29. The wound treatment system of claim 21, wherein the tab-seal is connected to the ampoule via a hinge.
